SEQUENCE LISTING (7 sequences)

SEQ ID NO: 1 - DNA, 2364 bp, Rattus norvegicus
tcggcacgagacgcggagaaaatggcggcaggggtcgaagcggcggccgaagtggcagcgacagagcccaaaatggaggaagagagcggcgcgccctgcgtgccgagcggcaacggagctccggtcccgaagggtgaagaacgacctactcagaatgagaagaggaaggagaaaaacataaaaagaggaggcaatcgctttgagccatatgcaaacccaactaaaagatacagagccttcattacaaatataccttttgatgtgaaatggcagtcacttaaagacctggttaaagaaaaagttggtgaggtaacatacgtggagctcttaatggacgctgaaggaaagtcaaggggatgtgctgttgttgagttcaagatggaggagagcatgaaaaaagctgccgaagttctaaacaagcatagtctgagtggaaggccactgaaagtcaaagaagatcctgatggtgaacatgcaaggagagcaatgcaaaaggctggaagacttggaagcacagtatttgtagcaaatctggattataaagttggctggaagaaactgaaagaagtatttagtatggctggtgtggtggtccgagcagacattctggaagataaagatgggaaaagtcgtggaataggcactgtgacttttgaacagtccattgaagctgtgcaagctatatctatgtttaatggccagttgctgtttgatagaccgatgcacgtcaagatggatgagagggctttaccaaagggagacttttttcctcctgaacgcccacagcaacttcctcatggacttggtggtatcggtatggggttaggacctggagggcagcctattgatgccaaccacctgaacaaaggcattggaatgggaaacctaggacctgcaggaatgggaatggaaggcataggatttggaataaataaaatcggaggcatggaaggaccctttggtggtggtatggaaaacatggggcgatttggatccgggatgaacatgggccggataaatgaaatcctaagtaatgcactgaagaggggagagatcattgcaaagcaaggaggaggtggagctggaggcagtgtccctgggatcgaaaggatgggccctggcattgaccgcattagcggtgctggcatggagcgcatgggcgcaggcttaggccatggcatggatcgagtgggctctgagattgagcgtatgggcctggtcatggaccgcatgggctcagttgagcgcatgggctccggcatcgagcgcatgggcccactaggccttgaccacatggcctccagtattgagcgcatgggccagaccatggaacgcattggttctggtgtggagcgcatgggtgccggtatgggcttcggcctggagcgcatggccgcacccattgaccgtgtgggccaaaccattgagcgcatgggctctggtgtagagcgcatgggtcctgccattgagcgaatgggcctaagcatggatcgaatggtgcccacaggcatgggggccggcctggagcgcatgggtcctgtgatggatcgtatggccaccggcctggagcgcatgggcgccaacaacctggagcgcatgggccttgagcgcatgggagccaacagtcttgagcgcatgggcctggaacgaatgggcgccaacagcctggaacggatgggccctgccatgggcccagcattgggcgctggtattgagcgaatgggcctggccatgggtggcgccggcggtgctagttttgaccgaaccattgagatggagcggggcaactttggaggaagcttcgcaggttcctttggcggagctggaggccatgcccctggagtagccaggaaggcctgccagatatttgtgagaaatctcccatttgattttacatggaagatgctaaaggacaaattcaatgaatgtggccacgtgctgtacgccgacatcaagatggagaacggaaagtccaaggggtgcggtgtggttaagtttgagtctccagaggtggctgagagagcctgccggatgatgaatggcatgaagctgagtggccgagagattgatgttcgaattgatagaaacgcttaagcagttgccttttttaaacatcagtaccagacctctgaatttgtattttttcttgttaaccattttaatttgttggctggatgtataaagatgtttaaaaaattcagttgctttttggggtaatttgaattaattttttaatgattggggttccatttgactgtttgcattgagattgcaatgtgcgcaattttttttgtagttgtggcatcttgttgacatcgaatatgactttgataataaataccagttcctgaaaaaaaaaaaaaa

SEQ ID NO: 2 - protein, 697 aa, Rattus norvegicus
SARDAEKMAAGVEAAAEVAATEPKMEEESGAPCVPSGNGAPVPKGEERPTQNEKRKEKNIKRGGNRFEPYANPTKRYRAFITNIPFDVKWQSLKDLVKEKVGEVTYVELLMDAEGKSRGCAVVEFKMEESMKKAAEVLNKHSLSGRPLKVKEDPDGEHARRAMQKAGRLGSTVFVANLDYKVGWKKLKEVFSMAGVVVRADILEDKDGKSRGIGTVTFEQSIEAVQAISMFNGQLLFDRPMHVKMDERALPKGDFFPPERPQQLPHGLGGIGMGLGPGGQPIDANHLNKGIGMGNLGPAGMGMEGIGFGINKIGGMEGPFGGGMENMGRFGSGMNMGRINEILSNALKRGEIIAKQGGGGAGGSVPGIERMGPGIDRISGAGMERMGAGLGHGMDRVGSEIERMGLVMDRMGSVERMGSGIERMGPLGLDHMASSIERMGQTMERIGSGVERMGAGMGFGLERMAAPIDRVGQTIERMGSGVERMGPAIERMGLSMDRMVPTGMGAGLERMGPVMDRMATGLERMGANNLERMGLERMGANSLERMGLERMGANSLERMGPAMGPALGAGIERMGLAMGGAGGASFDRTIEMERGNFGGSFAGSFGGAGGHAPGVARKACQIFVRNLPFDFTWKMLKDKFNECGHVLYADIKMENGKSKGCGVVKFESPEVAERACRMMNGMKLSGREIDVRIDRNA

SEQ ID NO: 3 - protein, 739 aa, Rattus norvegicus
  UNSURE (1)..(20): masked low complexity region; undetermined amino acid
XXXXXXXXXXXXXXXXXXXXTEIKMEECGSAPGVPSGNGAPGPKGEGERPAQNEKRKVKNIKRGGNRFEPYANPTKRYRAFITNIPFDVKWQSLKDLVKEKVGEVTYVELLMDAEGKSRGCAVVEFKMEESMKKAAEVLNKHSLSGRPLKVKEDPDGEHARRAKQKVMAXXXXXXXXXXXXXXXXXXXXXXXXXXXXXXXXHALQAGRLGSTVFVANLDYKVGWKKLKEVFSMAGVVVRADILEDKDGKSRGIGTVTFEQSIEAVQAISMFNGQLLFDRPMHVKMDERALPKGDFFPPERPQQXXXXXXXXXXXXXXXXXXIDANHLNKGIGMGNIGPAGMGMEGIGFGINKXXXXXXXXXXXXXXXXRFGSGMNMGRINEILSNALKRGEIIAKQXXXXXXXSVPGIERMGPGIDRLGGAGMERMGAGLGHGMDRVGSEIERMGLVMDRMGSVERMGSGIERMGPLGLDHMASSIERMGQTMERIGSGVERMGAGMGFGLERMAAPIDRVGQTIERMGSGVERMGPAIERMGPSMERMVPAGMGAGLERMGPVMDRMATGLERMGANNLERMGLERMGANSLERMGLERMGANSLERXXXXXXXXXXXXXIERXXXXXXXXXXXXSFDRAIEMERGNXXXXXXXXXXXXXXHAPGVARKACQIFVRNLPFDFTWKMLKDKFNECGHVLYADIKMENGKSKGCGVVKFESPEVAERACRMMNGMKLSGREIDVRIDRNA

SEQ ID NO: 4 - protein, 7 aa, Rattus norvegicus
KVGEVTY

SEQ ID NO: 5 - protein, 5 aa, Rattus norvegicus
PELPK

SEQ ID NO: 6 - DNA, 20 bp, Artificial Sequence
  Description of Artificial Sequence Primer
ggaaggccactgaaagtcaa

SEQ ID NO: 7 - DNA, 20 bp, Artificial Sequence
  Description of Artificial Sequence Primer
tccacgacttttcccatctt

(19) United States
(12) Patent Application Publication
(10) Pub. No.: US 2005/0058651 A1
(43) Pub. Date: Mar. 17, 2005

(54) Compositions and methods to prevent metastasis from primary malignancies

(76) Inventors: Peter Thomas, Pembroke,, MA (US); Olga Bajenova, Somerville, MA (US)

Correspondence Address: Ronald I Eisenstein, Nixon Peabody, 101 Federal Street, Boston, MA, 02110, US

(21) Appl. No.: 10/468,388

(22) PCT Filed: Mar. 21, 2002
(86) PCT No.: PCT/US02/08569

(30) Foreign Application Priority Data
Mar. 21, 2001 (US) 60277694

(51) Int. Cl. C12Q 1/68; G01N 33/574; C07H 21/04; A61K 39/395; C12N 9/64

(52) U.S. Cl. 424/155.1; 514/44; 435/6; 435/7.23; 435/69.1; 435/226; 435/320.1; 435/325; 530/388.26; 536/23.2

(57) ABSTRACT

We have now discovered a new method for treating carcinoembriyonic antigen (CEA) associated cancers. This involves blocking a protein expressed by the hnRNP M4 gene (preferably the human hnRNP M4 gene). We identified and isolated a liver-derived recombinant cDNA clone, termed heterogeneous nuclear RNA binding protein M4 (hnRNP M4) SEQ ID NO: 1, from rat macrophages Kupffer cells (KC) that encodes a novel protein interacting with CEA molecules and is 91% homologous with the deletion mutant of the human hnRNP M4 gene (# U32577). The novel protein is, hereinafter, considered as one protein population with the human homologue and referred to herein as hnRNP M4 CEA receptor, or hnRNP M4.

### GOVERNMENT FUNDING

This invention was made with government support under CA 74941 awarded by the National Institutes of Health. The government has certain rights in the invention.

### BACKGROUND

1. Field of the Invention

The present invention relates to carcinoembryonic antigen (CEA) molecules generally and metastasis of malignant cells. More specifically, the present invention relates to a method for treating CEA-associated cancers.

2. Background of the Invention

The liver is a common site for metastasis from various forms of primary malignancies. Both experimental and clinical results reveal that the presence of CEA enhances liver metastasis from colorectal carcinoma cells (1, 2). Increased amount of CEA in the serum correlates with the development of metastatic recurrence after the surgical removal of the primary tumor. In malignant conditions, the reported incidence of elevated serum CEA level ranges from 9% in testicular, ovary, lung, pancreatic and thyroid carcinomas to more than 50% in the metastatic colorectal carcinomas. CEA is an FDA approved tumor marker in the management of metastatic colon and breast cancers (3).

It has been previously shown that CEA production of human colorectal cancer cell lines directly correlates with the metastatic potential (4, 5). Poorly metastatic colon cancer cell lines become highly metastatic when transfected with the cDNA coding for CEA (6). As a member of the immunoglobulin supergene family, CEA is involved in the intercellular recognition and may facilitate attachment of colorectal carcinoma cells to sites of metastasis. In an experimental metastasis model of colorectal carcinoma in athymic nude mice, systemic injection of CEA enhanced experimental liver metastasis and implantation in liver by weakly metastatic tumor cells (7).

The mechanism by which CEA causes enhancement of metastasis is largely unknown. We have shown earlier that CEA is rapidly cleared from the circulation of experimental animals, accumulates in the liver and is endocytosed in vitro by KC (8). This uptake is independent of carbohydrates and is mediated by an 80 kD protein (9). The structure and the sequence of this protein is unknown. We have further shown that CEA is recognized by this binding protein through a five amino acid sequence, Pro-Glu-Leu-Pro-Lys (PELPK), located at the hinge region (amino-acids 108-112) between the N-terminal and the first immunoglobulin loop domain in the CEA sequence (10), and that the CEA binding to Kupffer cells initiates series of signaling events that lead to the tyrosine phosphorylation (23) and induction of IL-1α, IL-6, IL-10 and TNF-α cytokines (24). Molecular modeling studies have suggested that this region is exposed on the surface of the molecule (P. A. Bates, personal communication).

In order to successfully treat a multitude of malignant conditions, it is necessary to prevent the development of metastasis from cancerous cells after the treatment or removal of the primary malignancy. Therefore, there is a need to elucidate the mechanism by which CEA initiates series of signaling events that lead to metastasis from cancerous cells to healthy tissues, e.g., liver.

### SUMMARY OF THE INVENTION

We have now discovered a new method for treating carcinoembryonic antigen (CEA) associated cancers. This involves blocking a protein expressed by the hnRNP M4 gene (preferably the human hnRNP M4 gene). We identified and isolated a liver-derived recombinant cDNA clone, termed heterogeneous nuclear RNA binding protein M4 (hnRNP M4) SEQ ID NO: 1, from rat macrophages Kupffer cells (KC) that encodes a novel protein interacting with CEA molecules and is 91% homologous with the deletion mutant of the human hnRNP M4 gene (# U32577). The novel protein is, hereinafter, considered as one protein population with the human homologue and referred to herein as hnRNP M4 CEA receptor, or hnRNP M4.

We have surprisingly discovered that this gene product is the receptor for CEA. For example, transfection of rat hnRNP M4 cDNA into mouse macrophage cell line p388D1 resulted in CEA binding. To isolate the novel CEA receptor we used two approaches: screening of a KC cDNA library with the specific antibody and the yeast 2-hybrid system for the protein interaction using as a bait N-terminal part of the CEA encoding the binding sequence. Both techniques resulted in the isolation of a deletion mutant (amino acids 158-197) of the rat and human hnRNP M4 proteins. Thus, the new rodent clone is the rat homologue of the human hnRNP M4 (# U32577). The full-length cDNA is a 2351 bp complete ORF with the polyadenylation signal AATAAA (SEQ ID NO: 3) and a termination polyA tail. The mRNA shows ubiquitous tissue expression as a 2.4 kb transcript. The deduced amino acid sequence comprised a 78-kD membrane protein with 3 putative RNA-binding domains, arginine-methionine-glutamine rich C-terminus and 3 potential membrane spanning regions. Computer assisted evaluation of the hnRNP M4 sequence revealed a motif for tyrosine phosphorylation (KVGEVTY, SEQ ID NO: 4), 7 potential protein kinase C phosphorylation sites, 11 casein kinase 11 phosphorylation sites, two glucosaminoglycan attachment sites and 15 N-myristoylation sites. When hnRNP M4 protein is expressed in pGEX3T-4 vector system in E. coli it binds ¹²⁵I labeled CEA in a Ca²⁺-dependent fashion. These data provide an evidence for a new function of hnRNP M4 protein as a CEA binding protein.

To further study the interaction between the peptide sequence PELPK (SEQ ID NO: 5) and rat Kupffer cells, the CEA binding protein was purified using a combination of gel filtration, preparative polyacrylamide gel electrophoresis and affinity chromatography on CEA sepharose (11). A polyclonal antibody to the rat 80-kD protein was produced in mice that blocks both CEA and PELPK-albumin uptake by isolated rat Kupffer cells and shows a high degree of specificity for the rat 80-kDa protein by FACS analysis and Western blotting (11).

The present invention further provides DNA segments encoding the hnRNP M4 receptor proteins.

The present invention also provides an isolated DNA encoding a protein comprising the amino acids as set forth in SEQ ID NO: 2, as well as an isolated protein comprising the amino acids as set forth in SEQ ID NO: 2. Antibodies directed to amino acids of SEQ ID NO: 2, or a protein comprising amino acids of SEQ ID NO: 2 are also included. A DNA segment comprising the nucleotides as set forth in SEQ ID NO: 1 is further provided.

The present invention further provides assays for expression of the RNA and protein products of the DNA of the present invention to enable determining whether abnormal expression of such DNA is involved with a particular disease, e.g., cancer.

The present invention also provides antibodies, either polyclonal or monoclonal, specific to a unique portion of the receptor protein; a method for detecting the presence of a receptor ligand that is capable of either activating or down-regulating, i.e., modulating, the receptor protein; a method of screening potential ligand analogs for their ability to modulate the receptor protein; and procedures for targeting a therapeutic drug to cells having a high level of the receptor protein.

The present invention also provides binding assays that permit the ready screening for molecules that affect the binding of the receptor and its ligands.

The present invention further provides use of the receptor for intracellular or extracellular targets to affect binding. Intracellular targeting can be accomplished through the use of intracellularly expressed antibodies referred to as intrabodies. Extracellular targeting can be accomplished through the use of receptor specific antibodies. Additionally, the soluble form of the receptor can be used as a receptor decoy or aptamer to inhibit binding.

The present invention also provides use of antisense technology to affect binding of the CEA receptor and its ligands by designing an antisense nucleic acid molecule which is complementary to a nucleic acid molecule encoding the CEA receptor.

The present invention also provides an assay to determine the presence or absence of the receptors that can be used as a diagnostic or prognostic tool to identify the presence or stage of differentiation of tissue, e.g., tumor tissue.

Finally, a nucleic acid of the invention could be used to select a catalytic RNA having a specific ribonuclease activity from a pool of RNA molecules. RNA-mediated interference (RNAi) may also be used.

### BRIEF DESCRIPTION OF THE FIGURES

FIG. 1 illustrates SEQ ID NO: 1 which is the nucleotide sequence of rat hnRNP M4 cDNA. This cDNA was isolated from rat KC as encoding the CEA binding protein. It is a full-length sequence that consists of a 5′ end nontranslated region of 21 bp and an open reading frame (ORF) that starts from the initiation sequence AAAATGG. The initiation, termination codons and a polyA tail shown in bold. The two N-terminal RNA-binding domains followed by methionine-arginine-glycine rich region and C-terminal RBD-3 shown as boxes. Inside the RBD-1 and RBD-2 the regions of homology with the Mycf-2 transcription factor are underlined. The linker sequence between the RBD-1 and RBD-2 containing 158-197 deletion shown in italic. The double underlined amino acids correspond the primer's sequences (5′-GGAAGGCCACTGAAAGTCAA (SEQ ID NO: 6), 3′-TCCACGACTTTTCCCATCTT (SEQ ID NO: 7)) employed for RT-PCR amplification. The GeneBank accession No. for the sequence is U32577.

FIGS. 2(a) and 2(b) show the predicted amino acid sequence of hnRNP M4 cDNA. FIG. 2(a) shows the domain structure of rat hnRNP M4 protein. The protein composed of 2 N-terminal RNA-binding domains followed by methionine-arginine-glycine rich region and C-terminal RBD-3 shown as boxes. The potential transmembrane regions are shown as black boxes. The numbers above correspond to the amino acids. FIG. 2(b) shows the posttranslational modification sites of rat hnRNP M4 protein. The predicted tyrosine kinase phosphorylation site, KVGEVTY (SEQ ID NO: 4), (aa 100-106) is shown between the arrows. The (#) symbols indicate 2 potential N-linked glycosylation sites (aa 36-39; 379-382). The (o) symbols indicate 7 potential protein kinase C phosphorylation sites (aa 1-3; 74-76; 92-94; 130-132; 144-146; 632-634; 685-687). The (*) symbols indicate 11 potential casein kinase II phosphorylation sites (aa 1-4; 50-53; 92-95; 105-108; 399-402; 419-422; 434-437; 448-451; 480-483; 520-523; 685-688). The (+) symbols indicate 15 potential casein kinase II phosphorylation sites (aa 11-16; 270-275; 290-295; 305-310; 322-327; 331-336; 37-362; 363-368; 387-392; 454-459; 503-508; 575-580; 595-600; 603-608; 681-686.).

FIG. 3 illustrates SEQ ID NO: 2 which is a comparison of published (Kafasla P., et al., Biochem J.; 2000, 350: 495-503) and deduced from cDNA amino acid sequences of rat hnRNP M4 protein. Amino acids are shown in single letter code. The sequences are indicated on the top and the amino acid numbers on the right and on the left sides. The consensus sequence shown in the middle represents the identical amino acids in both proteins.

FIG. 4 is a Western blot of rat hnRNP M4 protein expressed in E coli showing CEA binding. Rat cDNA corresponding to hnRNP M4 ORF was placed into a prokaryotic expression vector pGEX3T-4 allowing production of a GST-hnRNP M4 fusion protein. The Kupffer cell lysate and pGEX-4T-3/hnRNP M4 fusion proteins (2 clones) were subjected to SDS/PAGE and transferred into the PVDF membrane. This membrane was exposed to the soluble 1 μM CEA followed by the anti-CEA antibody. Similar to KC (line 1), hnRNP M4 protein (lines 2 and 3) binds to the soluble CEA and has a molecular weight 80 KD. Line 4 corresponds to a negative control that is a pGEX3T-4 vector without the hnRNP M4 cDNA.

FIGS. 5A-C show distribution of different splicing forms of hnRNP M4 protein in rat tissues. To determine whether the form with the deletion in a spacer region between the RBD-1 and RBD-2 is expressed in Kupffer cells we synthesized PCR primers that incorporated the deleted region as part of their product. Amplification will result in a larger PCR product for the full-length form (321 bp) and a shorter product (204 bp) for the deletion mutant. We examined a number of rat tissues. The results show that the deletion form encoding amino acids 158-197 of hnRNP M4 is distributed throughout all rat tissues examined (FIGS. 5A, 5B and 5C). The larger form is generally also present but in what seems to be lower copy numbers. Comparison of the cDNA sequences indicates that there are multiple subclasses of mRNA that arise by alternative pre-mRNA splicing. This suggests that multiple forms of the hnRNP M4 protein may exist, possibly with different functions in vivo. Both mRNA forms (with and without the deletion) were characteristic to the rat KC. Only the short form was determined in human KC.

FIG. 6 shows that the expression of the 80 kD CEA receptor and the ability to uptake CEA are cell specific and perhaps tissue restricted to KC and alveolar macrophages. To elucidate whether the macrophage cell lines can uptake soluble CEA, in vitro experiments with ¹²⁵I, labeled CEA were performed. Previously we have reported that LPS treatment can stimulate the CEA uptake (Toth CA et al, J. Leukocyte Biology. 1989; 45: 370-376.). A very high level of CEA uptake characteristic to KC was used as a positive control. The macrophage cell lines pretreated with LPS (1 μg/ml, 1 hour) and without LPS exposure were incubated with the iodine labeled CEA (5 μg/ml) for 15, 30, 45 and 60 minutes. Comparatively, none of the macrophage cell lines was able to uptake CEA with or without LPS pretreatment. We have shown earlier that only freshly isolated lung alveolar macrophages express 80 kD protein and rapidly endocytosed CEA in a similar manner to KC. These data suggest that the expression of the 80 kD CEA receptor and the ability to uptake CEA are cell specific and perhaps are tissue restricted to KC and alveolar macrophages.

FIG. 7 illustrates that transfection of hnRNP M4 cDNA can initiate CEA binding in P388D1 macrophage cell line. To determine the mechanism underlying the intracellular signaling associated with KC activation and CEA binding, the hnRNP M4 cDNA in the pBK-CMV expression vector was introduced into the CEA non-responsive macrophage cell lines. The hnRNPM4 transcription in this system was driven by the CMV promoter. The two mouse (P388D1 and IC21) and a rat macrophage cell line (CRL2192) that did not bind CEA were transfected with the hnRNP M4/PBK-CMV expression vector. As shown in FIG. 7, transient transfection of bnRNPM4 (48 hours) in P388D1 cells results in CEA binding. The uptake was increased with increasing time of exposure and concentration of the hnRNP M4 plasmid DNA. An approximately 5 times increase in CEA uptake was observed on transfecting 10 g of hnRNP M4/pBK-CMV plasmid. This fact implies that hnRNP M4 is involved in CEA metabolism in P388D1 macrophages. Interestingly, P388D1, but not IC21 or CRL2192 cells were able to uptake CEA. At present, the reasons for lack of response by IC21 and CRL2192 cells is not known. It is possible that these cells may not have the regulatory factors that can induce hnRNP M4 gene expression as a result of differentiation and tissue specificity.

FIG. 8 is a schematic of CEA internalization by Kupffer cells.

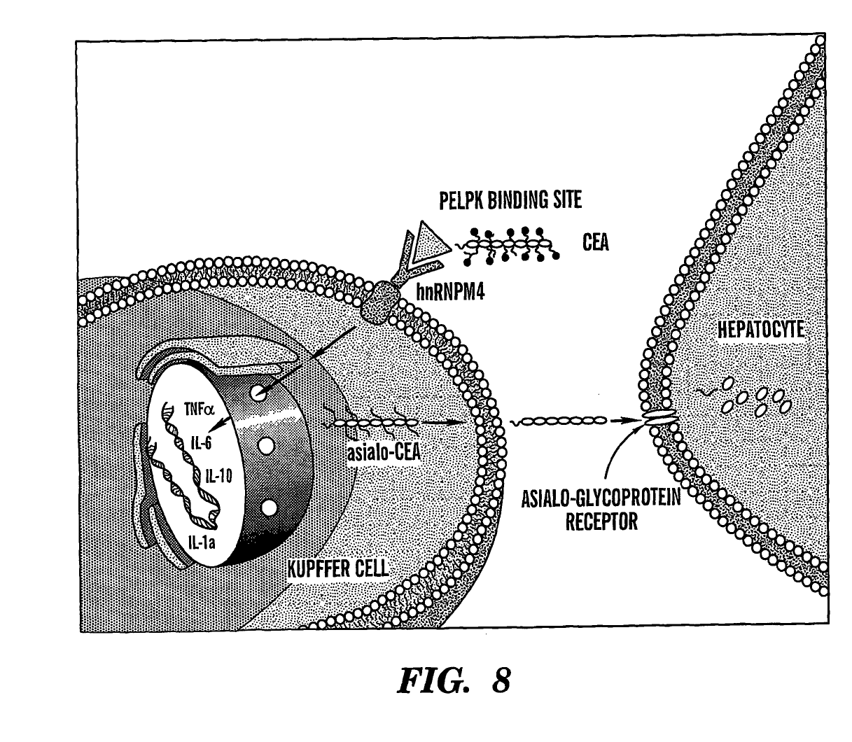

### DETAILED DESCRIPTION

The present invention provides a method of inhibiting CEA-associated process of cancer metastasis by identifying and targeting the human hnRNP M4 receptor that mediates this process. The novel receptor for CEA is the hnRNP M4 protein which was isolated by means of expression cloning using a mouse polyclonal 80 kD protein specific antiserum. The novel receptor for CEA from the rat KC is homologous to the deletion mutant of the human hnRNP M4 gene.

The cDNA displays features similar to that of 80 kD KC CEA receptor by four independent criteria: (1) By SDS/PAGE the molecular masses of both the recombinant and the cellular proteins were found to be similar corresponding 78-80 kD; (2) The rat hnRNP M4 fusion protein expressed in E. coli binds CEA and this interaction is a Ca⁺⁺ dependent process; (3) Transfection of rat hnRNP M4 cDNA in P388D1 mouse macrophages results in CEA uptake; (4) The same gene was identified for the binding protein in both rat and human liver using two different approaches: screening with the anti-80 kD antibody and two-hybrid assay for the protein interactions.

Earlier we have shown that the CEA binding to KC initiates series of signaling events that lead to the induction of IL-1α, IL-6, IL-10 and TNFE-α cytokines (24). Data presented in this study shows that the hnRNP M4 protein can bind CEA. Without wishing to be bound by theory, we predict that it is involved in a multi-protein complex that can recognize CEA on the surface of KC. Earlier it was shown that the increase in cytokines production is associated with tyrosine phosphorylation (23). The analysis of conserved domains of the encoded protein by Swiss-Prot database revealed that hnRNP M4 has a tyrosine internalization signal, KVGEVTY (SEQ ID NO: 4), aa 100-106. Enhancement in tyrosine phosphorylation after receptor activation is considered an important signaling event leading to cellular responses. For example, the hnRNP K protein is tyrosine phosphorylated in vitro by Src and Lck that regulates in vivo K-protein-protein and K-protein-RNA interactions by changing recruitment of signaling effectors (33). HnRNP K has a diverse repertoire of molecular partners including G coupled receptor protein, tyrosine and serine/threonine kinases as well as the proto-oncoprotein Vav (34). It was shown that of hnRNP A2 and hnRNP C1 proteins in the liver can be phosphorylated and this process is modulated by calmodulin (35). In addition to a tyrosine kinase phosphorylation site, the hnRNP M4 possesses 7 potential protein kinase C that can also play an important role in the signal transduction pathways.

Similar to hnRNP K the cell distribution pattern of hnRNP M4 depends on the tissue examined. In Hela cells this protein is evenly present throughout the nucleus and the cytoplasm (19). In contrast, in mice's embryos the protein is localized predominantly in the nucleus (20). The hnRNP M4 protein can also form complexes with hnRNP K and hnRNP A1 shuttling proteins (34, 36).

We therefore discovered a new physiological role for the hnRNP M4 protein as a CEA binding protein in Kupffer cells and also in lung alveolar macrophages (32). One of the mechanisms of how the hnRNPs can carry different functions is structural rearrangement and the appearance of different splicing forms (15).

The present invention shows that at least 3 splicing forms of hnRNP M4 are ubiquitously expressed in rat and human tissues. Two low molecular weight mRNA forms (with and without the deletion of aa 158-197) were characteristic to rat KC. Only the short form is transcribed in human KC. When rat cDNA with the deletion was translated in the prokaryotic system it was able to bind with ¹²⁵I labeled CEA or PELPK (SEQ ID NO: 5) albumin conjugate in the presence of 10 mM Ca⁺⁺. This binding was abolished in the presence of 10 mM EDTA. This data shows that the low molecular weight form of the protein that has a deletion of aa 158-197 in the spacer region between RBD-1 and RBD-2 can function as a CEA receptor.

The signaling pathway of KC activation by CEA leading to the enhancement of metastasis is not known. Based on the present invention, several mechanisms of cytokine regulation by CEA can be envisioned. First, structural findings reveal that hnRNP M4 receptor has a potential to be involved in signaling pathways and to be modified by a variety of enzymes. Second, high level of homology of N-terminal RBD-1 and RBD-2 domains with the Myef-2 transcription factor suggests a role of hnRNP M4 as a transcription factor. By participating in transcription and regulating the promoter function of the genes, hnRNP M4 could modulate the cytokine production. Third, the hnRNPs can also effect the cytokine production through a direct binding with nRNA and control of mRNA stability. For many protooncogenes, lymphokines, and cytokines, a common feature is the existence of A+U-rich elements (15). It was shown that all four hnRNP M proteins bind to poly (U) stretches in high salt conditions (1M NaCl) (17, 18, 19). Uridylate stretches also found in regulatory regions of RNAs such as the 3′ splice site of introns appear to be common targets for RNA-binding proteins (15).

In support of our previous findings (32), the present invention teaches that the ability to take up CEA is tissue specific. Several macrophage cell lines: CRL2192, P388D, IC21, PH1 and raw 264.7 were analyzed and none of them were as effective in taking up CEA as KC. We transfected these cells with the expression pBK-CMV/hnRNP M4 vector to elucidate whether the hnRNP M4 protein expression can result in CEA uptake. Only one cell line, a mouse macrophage cell line p388D1, initiated CEA binding in transient transfection assay. This is an important evidence of the role of hnRNP M4 in CEA metabolism. A developed system represents an in vitro model to study and to reconstitute the signaling events that occur in vivo in KC.

Further investigations by using deletions of specific domains within the gene and examining the effects on specific functions will result in the identification of CEA (PELPK (SEQ ID NO: 5)) binding domains and the structure-function relationships associated with the CEA binding. This model also allows examination of downstream signaling events and protein domains needed to initiate the receptor mediated endocytosis and to induce the cytokine secretion in macrophages by CEA. Identifying the nature of the biochemical differences between KC and macrophage cell lines, which allow hnRNP M4 function more efficiently in some cell lines than others, will help our understanding of the complex regulatory mechanisms involved in the CEA receptor expression and CEA binding.

CEA is not only a serum marker to monitor metastasis but is also a target for therapeutic treatment. Conventional treatments of metastatic colon cancer such as chemo- and radiotherapy are lacking specificity and have dose limitations due to toxicity in normal tissues. Anti-CEA antibodies are widely used for localization of colorectal carcinoma in radioinimunoguided surgery (37), radioimmunotherapy (38) and anti-CEA antibody-directed cytokine targeting (39). CEA is also a potential T-cell target in antigen-specific vaccination-based cancer therapy (40). Development of specific antibodies and vaccines to the CEA receptor seems as a more straightforward approach to study and prevent metastasis, e.g., hepatic metastasis.

The receptor of the present invention may also be used diagnostically. Determining the level of this receptor in individuals can be an important tool in determining whether an individual is at a greater risk for developing metastasis of cancerous cells post treatment or post surgery. This knowledge can be used in determining a more effective treatment plan for that individual.

The determination of the number of receptors present on the cells of an individual can readily be accomplished by standard means, for example, using FACS analysis or analysis of RNA levels. The level can be compared to a reference level, which can be determined by standard means. These assays are further discussed below.

Another preferred embodiment of this invention is in the diagnosis of diseases associated with this receptor.

Using any suitable technique known in the art, such as Northern blotting, quantitative PCR, reverse transcriptase PCR, etc. the nucleotide sequences of the receptors or fragments thereof can be used to measure levels of receptor RNA expression.

Alternatively, the antibodies of the invention can be used in standard techniques such as Western blotting to detect the presence of cells expressing receptors and using standard techniques, e.g. FACS or ELISA, to quantify the level of expression.

One can treat diseases associated with the expression of the receptors of the present invention by blocking receptor/ligand interaction. This can be accomplished by a range of different approaches. For example, antibodies, aptamers, decoys, small molecules, antagonists, etc. One preferred approach is the use of antibodies to these receptors. Antibodies specifically binding to amino acids of SEQ ID NO: 2 are preferred. Antibodies to these receptors can be prepared by standard means. For example, one can use single chain antibodies to target these receptors.

An alternative strategy is to use receptor decoys. For example, one could prepare a decoy comprising the portion of the receptor present on the exterior of the cell membrane. Another strategy is to prepare soluble forms of these receptors. This can be done by standard means including using PCR to clone a gene, site-directed mutagenesis to make changes in the structure, deletions to make fragments, etc. as discussed below.

Compounds that affect this receptor/ligand interaction can be directly screened for example using a direct binding assay. For example, the compound of interest can be added before or after the addition of the labeled ligand and the effect of the compound on binding can be determined by comparing the degree of binding in that situation against a base line standard with that ligand, not in the presence of the compound. The binding assay can be adapted depending upon precisely what is being tested.

The present invention also provides use of antisense technology to affect binding of the CEA receptor and its ligands. Antisense technology can be used to control gene expression through triple-helix formation or antisense DNA or RNA, both of which methods are based on binding of a polynucleotide to DNA or RNA. An antisense nucleic acid molecule which is complementary to a nucleic acid molecule encoding the CEA receptor can be designed based upon the isolated nucleic acid molecules encoding the receptor provided by the invention.

An antisense nucleic acid molecule can comprise a nucleotide sequence which is complementary to a coding strand of a nucleic acid, e.g. complementary to an mRNA sequence, constructed according to the rules of Watson and Crick base pairing, and can hydrogen-bond to the coding strand of the nucleic acid. The antisense sequence complementary to a sequence of an mRNA can be complementary to a sequence in the coding region of the mRNA or can be complementary to a 5′ or 3′ untranslated region of the mRNA.

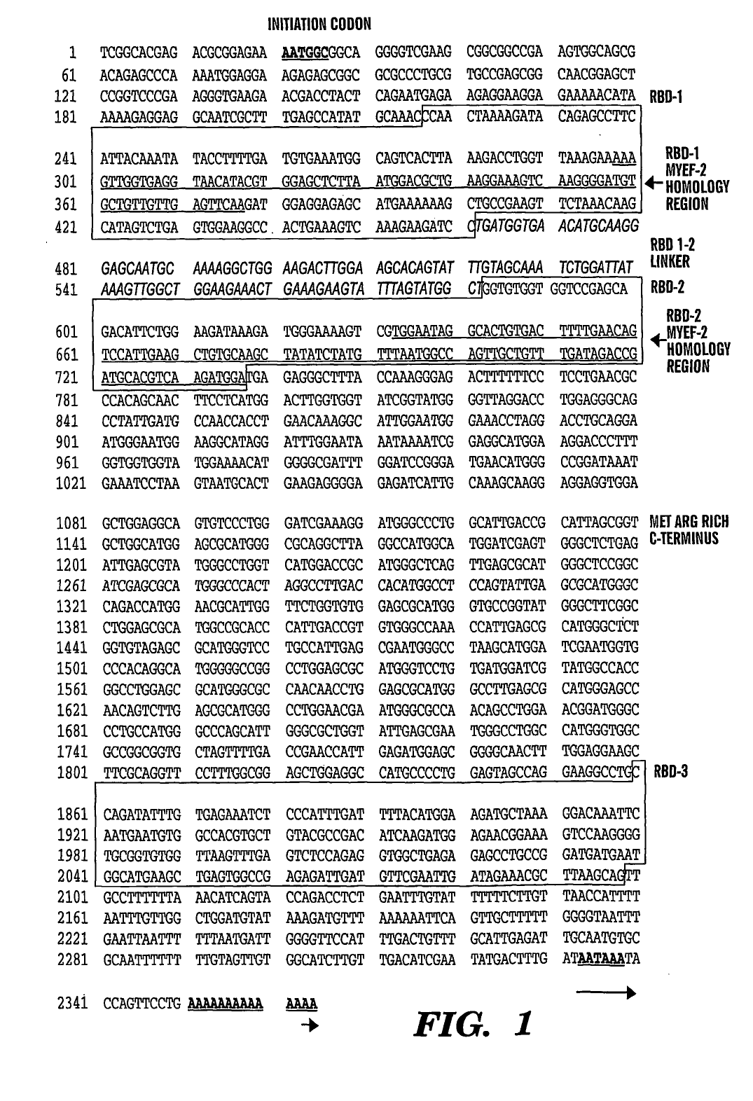

Furthermore, an antisense nucleic acid can be complementary in sequence to a regulatory region of the gene encoding the mRNA, for instance a transcription initiation sequence or regulatory element. Preferably, an antisense nucleic acid complementary to a region preceding or spanning the initiation codon or in the 3′ untranslated region of an mRNA is used. An antisense nucleic acid can be designed based upon the nucleotide sequence of SEQ ID NO: 1 (shown in FIG. 1). A nucleic acid is designed which has a sequence complementary to a sequence of the coding or untranslated region of the shown nucleic acid. Alternatively, an antisense nucleic acid can be designed based upon sequences of the CEA receptor, which can be identified by screening a genomic DNA library with an isolated nucleic acid of the invention. For example, the sequence of an important regulatory element can be determined by standard techniques and a sequence which is antisense to the regulatory element can be designed.

The antisense nucleic acids and oligonucleotides of the invention can be constructed using chemical synthesis and enzymatic ligation reactions using procedures known in the art. The antisense nucleic acid or oligonucleotide can be chemically synthesized using naturally occurring nucleotides or variously modified nucleotides designed to increase the biological stability of the molecules or to increase the physical stability of the duplex formed between the antisense and sense nucleic acids e.g. phosphorothioate derivatives and acridine substituted nucleotides can be used. Alternatively, the antisense nucleic acids and oligonucleotides can be produced biologically using an expression vector into which a nucleic acid has been subcloned in an antisense orientation (i.e. nucleic acid transcribed from the inserted nucleic acid will be of an antisense orientation to a target nucleic acid of interest). The antisense expression vector is introduced into cells in the form of a recombinant plasmid, phagemid or attenuated virus in which antisense nucleic acids are produced under the control of a high efficiency regulatory region, the activity of which can be determined by the cell type into which the vector is introduced, For a discussion of the regulation of gene expression using antisense genes see Weintraub, H. et al., Antisense RNA as a molecular tool for genetic analysis, Reviews—Trends in Genetics, Vol. 1 (1) 1986.

A nucleic acid of the invention could be used to select a catalytic RNA having a specific ribonuclease activity from a pool of RNA molecules. See for example Bartel, D. and Szostak, J. W. Science 261: 1411-1418 (1993). RNA-mediated interference (RNAi) (Fire, et al., Nature 391: 806-811, 1998) may also be used.

Aptamers can be produced using the methodology disclosed in a U.S. Pat. No. 5,270,163 and WO 91/19813.

The DNA segments according to this invention are useful for detection of expression of the receptors in tissues, as described in the Examples below. Therefore, in yet another aspect, the present invention relates to a bioassay for determining the amount of receptor mRNA in a biological sample comprising the steps of i) contacting that biological sample with a nucleic acid isolate consisting essentially of a nucleotide sequence that encodes the receptor or a unique portion thereof, e.g., such the nucleotide sequence of SEQ ID NO: 1 (shown in FIG. 1), under conditions such that a nucleic acid:RNA hybrid molecule, such as a DNA:RNA hybrid molecule, can be formed; and ii) determining the amount of hybrid molecule present, the amount of hybrid molecule indicating the amount of receptor raRNA in the sample. Findings described in the Examples, below, indicate that increased expression of the receptor of the present invention, as detected by this method of this invention, plays a critical role in the success of treatment of some human malignancies, e.g., colon and breast cancer.

Of course, it will be understood by one skilled in the art of genetic engineering that in relation to production of polypeptide products, the present invention also includes DNA segments having DNA sequences other than those in the present examples that also encode the amino acid sequence of the polypeptide product of the receptor gene. For example, it is known that by reference to the universal genetic code, standard genetic engineering methods can be used to produce synthetic DNA segments having various sequences that encode any given amino acid sequence. Such synthetic DNA segments encoding at least a portion of the amino acid sequence of the polypeptide product of the receptor gene also fall within the scope of the present invention. Further, it is known that different individuals may have slightly different DNA sequences for any given human gene and, in some cases, such mutant or variant genes encode polypeptide products having amino acid sequences which differ among individuals without affecting the essential function of the polypeptide product. Still further, it is also known that many amino acid substitutions can be made in a polypeptide product by genetic engineering methods without affecting the essential function of that polypeptide. Accordingly, the present invention further relates to a DNA segment having a nucleotide sequence that encodes an amino acid sequence differing in at least one amino acid from the amino acid sequence of receptor, or a unique portion thereof, and having greater overall similarity to the amino acid sequence of the receptor than to that of any other polypeptide. The amino acid sequence of this DNA segment includes at least about 4 to 6 amino acids which are sufficient to provide a binding site for an antibody specific for the portion of a polypeptide containing this sequence.

The present invention further relates to a recombinant DNA molecule comprising a DNA segment of this invention and a vector. In yet another aspect, the present invention relates to a culture of cells transformed with a DNA segment according to this invention. These host cells transformed with DNAs of the invention include both higher eukaryotes, including animal, plant and insect cells, and lower eukaryotes, such as yeast cells, as well as prokaryotic hosts including bacterial cells such as those of E. coli and Bacillus subtills.

Various standard recombinant systems, such as those cited above as well as others known in the art, are suitable as well for production of large amounts of the novel receptor proteins using methods of isolation for receptor proteins that are well known in the art. Therefore, the present invention also encompasses an isolated polypeptide having at least a portion of the amino acid sequence of SEQ ID NO: 2.

The isolated nucleotide sequences and isolated polypeptides of the invention encoding receptors can be mutagenized by any of several standard methods including treatment with hydroxylamine, passage through mutagenic bacterial strains, etc. The mutagenized sequences can then be classified “wild type” or “non-wild type” depending whether it will still facilitate infectivity or not.

Mutagenized sequences can contain point mutations, deletions, substitutions, rearrangements etc. Mutagenized sequences can be used to define the cellular function of different regions of the receptors they encode. This information can be used to assist in the design of small molecules or peptides mimicking the interactive part of the receptor.

Another approach is to use small molecules that will selectively bind to one of the receptors. Such molecules and peptides can be synthesized by known techniques.

Another strategy is to express antibodies to these receptors in individuals intracellularly. This can be done by the method of Marasco and Haseltine set forth in WO94-02610 (PCT/US93/06735 filed Jul. 16, 1993) published Feb. 3, 1994.

In addition, additional compounds that bind to these receptors can readily be screened for. For example, one can select cells expressing high numbers of these receptors, plate them; e.g. add labeled ligand and screen for compounds or combinations of compounds that will interact with, e.g. binding of, these receptors by standard techniques. Alternatively, one can use known techniques to prepare cells that will express these receptors and use those cells in drug screens.

One can also prepare cell lines stably expressing the receptors. Such cells can be used for a variety of purposes including an excellent source of antigen for preparing a range of antibodies using techniques well known in the art.

Therapeutic and Pharmaceutic Compositions.

An exemplary pharmaceutical composition is a therapeutically effective amount of a decoy, antibody etc. that affects the ability of the receptor to bind ligand optionally included in a pharmaceutically-acceptable and compatible carrier. The term “pharmaceutically-acceptable and compatible carrier” as used herein, and described more fully below, includes (1) one or more compatible solid or liquid filler diluents or encapsulating substances that are suitable for administration to a human or other animal, and/or (ii) a system, such as a retroviral vector, capable of delivering the molecule to a target cell. In the present invention, the term “carrier” thus denotes an organic or inorganic ingredient, natural or synthetic, with which the molecules of the invention are combined to facilitate application. The term “therapeutically-effective amount” is that amount of the present pharmaceutical compositions which produces a desired result or exerts a desired influence on the particular condition being treated. Various concentrations may be used in preparing compositions incorporating the same ingredient to provide for variations in the age of the patient to be treated, the severity of the condition, the duration of the treatment and the mode of administration.

The term “compatible”, as used herein, means that the components of the pharmaceutical compositions are capable of being commingled with a small molecule, nucleic acid and/or polypeptides of the present invention, and with each other, in a manner such that does not substantially impair the desired pharmaceutical efficacy.

Dose of the pharmaceutical compositions of the invention will vary depending on the subject and upon particular route of administration used. Dosages can range from 0.1 to 100,000 μg/kg per day, more preferably 1 to 10,000 Ag/kg.

By way of an example only, an overall dose range of from about, for example, 1 microgram to about 300 micrograms might be used for human use. This dose can be delivered at periodic intervals based upon the composition. For example on at least two separate occasions, preferably spaced apart by about 4 weeks. Other compounds might be administered daily.

Pharmaceutical compositions of the present invention can also be administered to a subject according to a variety of other, well-characterized protocols. For example, certain currently accepted immunization regimens can include the following: (i) administration times are a first dose at elected date; a second dose at 1 month after first dose; and a third dose at 5 months after second dose. See Product Information, Physician's Desk Reference, Merck Sharp & Dohme (2002) (e.g., Hepatitis B Vaccine-type protocol); (ii) Recommended administration for children is first dose at elected date (at age 6 weeks old or older); a second dose at 4-8 weeks after first dose; a third dose at 4-8 weeks after second dose; a fourth dose at 6-12 months after third dose; a fifth dose at age 4-6 years old; and additional boosters every 10 years after last dose. See Product Information, Physician's Desk Reference, Merck Sharp & Dohme (2002) (e.g., Diptheria, Tetanus and Pertussis-type vaccine protocols). Desired time intervals for delivery of multiple doses of a particular composition can be determined by one of ordinary skill in the art employing no more than routine experimentation.

The small molecules and polypeptides of the invention may also be administered per se or in the form of a pharmaceutically-acceptable salt. When used in medicine, the salts should be pharmaceutically acceptable, but non-pharmaceutically acceptable salts may conveniently be used to prepare pharmaceutically acceptable salts thereof and are not excluded from the scope of this invention. Such pharmaceutically acceptable salts include, but are not limited to, those prepared from the following acids: hydrochloric, hydrobromic, sulfuric, nitric, phosphoric, maleic, acetic, salicylic, p-toluene-sulfonic, tartaric, citric, methanesulphonic, formic, malonic, succinic, naphthalene-2-sulfonic, and benzenesulphonic.

Also, pharmaceutically acceptable salts can be prepared as alkaline metal or alkaline earth salts, such as sodium, potassium or calcium salts of the carboxylic acid group. Thus, the present invention also provides pharmaceutical compositions, for medical use, which comprise nucleic acid and/or polypeptides of the invention together with one or more pharmaceutically acceptable carriers thereof and optionally any other therapeutic ingredients. The compositions include those suitable for oral, rectal, intravaginal, topical, nasal, ophthalmic or parenteral administration, all of which may be used as routes of administration using the materials of the present invention. Other suitable routes of administration include intrathecal administration directly into spinal fluid (CSF), direct injection onto an arterial surface and intraparenchymal injection directly into targeted areas of an organ. Compositions suitable for parenteral administration are preferred. The term “parenteral” includes subcutaneous injections, intravenous, intramuscular, intrasternal injection or infusion techniques.

The compositions may conveniently be presented in unit dosage form and may be prepared by any of the methods well known in the art of pharmacy. Methods typically include the step of bringing the active ingredients of the invention into association with a carrier which constitutes one or more accessory ingredients.

Compositions of the present invention suitable for oral administration may be presented as discrete units such as capsules, cachets, tablets or lozenges, each containing a predetermined amount of the nucleic acid and/or polypeptide of the invention in liposomes or as a suspension in an aqueous liquor or non-aqueous liquid such as a syrup, an elixir, or an emulsion.

Preferred compositions suitable for parenteral administration conveniently comprise a sterile aqueous preparation of the molecule of the invention which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectibles.

Antibodies

The term “antibodies” is meant to include monoclonal antibodies, polyclonal antibodies and antibodies prepared by recombinant nucleic acid techniques that are selectively reactive with polypeptides encoded by eukaryotic nucleotide sequences of the present invention. The term “selectively reactive” refers to those antibodies that react with one or more antigenic determinants of the receptors and do not react with other polypeptides. Antigenic determinants usually consist of chemically active surface groupings of molecules such as amino acids or sugar side chains and have specific three dimensional structural characteristics as well as specific charge characteristics. Antibodies can be used for diagnostic applications or for research purposes.

For example, antibodies may be raised against amino-terminal (N-terminal) or carboxyl-terminal (C-terminal) peptides of a polypeptide encoded by the receptors.

One approach is to isolate a peptide sequence that contains an antigenic determinant for use as an immunogen. This peptide immunogen can be attached to a carrier to enhance the immunogenic response. Although the peptide immunogen can correspond to any portion of a polypeptide encoded by a eukaryotic nucleotide sequence of the invention, certain amino acid sequences are more likely than others to provoke an immediate response, for example, an amino acid sequence including the N- or C-terminus of a polypeptide encoded by a gene that contains nucleotide sequences of the invention. Preferably one can use a cell line expressing only the receptor, select those cells with the highest levels of expression and use the whole cell as an antigen.

For example, cDNA clone encoding a receptor or a fragment thereof may be expressed in a host using standard techniques (see above; see Sambrook et al., Molecular Cloning; A Laboratory Manual, Cold Spring Harbor Press, Cold Spring Harbor, N.Y.: 1989) such that 5-20% of the total protein that can be recovered from the host is the desired protein. Recovered proteins can be electrophoresed using PAGE and the appropriate protein band can be cut out of the gel. The desired protein sample can then be eluted from the gel slice and prepared for immunization. Alternatively, a protein of interest can be purified by using conventional methods such as, for example, ion exchange hydrophobic, size exclusion, or affinity chromatography.

Once the protein immunogen is prepared, mice can be immunized twice intraperitoneally with approximately 50 micrograms of protein immunogen per mouse. Sera from such immunized mice can be tested for antibody activity by immunohistology or immunocytology on any host system expressing such polypeptide and by ELISA with the expressed polypeptide. For immunohistology, active antibodies of the present invention can be identified using a biotin-conjugated anti-mouse immunoglobulin followed by avidin-peroxidase and a chromogenic peroxidase substrate. Preparations of such reagents are commercially available; for example, from Zymad Corp., San Francisco, Calif. Mice whose sera contain detectable active antibodies according to the invention can be sacrificed three days later and their spleens removed for fusion and hybridoma production. Positive supernatants of such hybridomas can be identified using the assays described above and by, for example, Western blot analysis.

To further improve the likelihood of producing an antibody as provided by the invention, the amino acid sequence of polypeptides encoded by a eukaryotic nucleotide sequence of the present invention may be analyzed in order to identify portions of amino acid sequence which may be associated with increased immunogenicity. For example, polypeptide sequences may be subjected to computer analysis to identify potentially immunogenic surface epitopes. Such computer analysis can include generating plots of antigenic index, hydrophilicity, structural features such as amphophilic helices or amphophilic sheets and the like.

For preparation of monoclonal antibodies directed toward polypeptides encoded by a eukaryotic nucleotide sequence of the invention, any technique that provides for the production of antibody molecules by continuous cell lines may be used. For example, the hybridoma technique originally developed by Kohler and Milstein (Nature, 256: 495-497, 1973), as well as the trioma technique, the human B-cell hybridoma technique (Kozbor et al., Immunology Today, 4:72), and the EBV-hybridoma technique to produce human monoclonal antibodies, and the like, are within the scope of the present invention. See, generally Larrick et al., U.S. Pat. No. 5,001,065 and references cited therein. Further, single-chain antibody (SCA) methods are also available to produce antibodies against polypeptides encoded by a eukaryotic nucleotide sequence of the invention (Ladner et al. U.S. Pat. Nos. 4,704,694 and 4,976,778).

The monoclonal antibodies may be human monoclonal antibodies or chimeric human-mouse (or other species) monoclonal antibodies. The present invention provides for antibody molecules as well as fragments of such antibody molecules.

Those of ordinary skill in the art will recognize that a large variety of possible moieties can be coupled to the resultant antibodies or to other molecules of the invention. See, for example, “Conjugate Vaccines”, Contributions to Microbiology and Immunology, J. M. Cruse and R. E. Lewis, Jr. (eds), Carger Press, New York, (1989), the entire contents of which are incorporated herein by reference.

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehydes, diazobenzenes and hexamethylene diamines. This listing is not intended to be exhaustive of the various classes of coupling agents known in the art but, rather, is exemplary of the more common coupling agents. (See Killen and Lindstrom 1984, “Specific killing of lymphocytes that cause experimental Autoimmune Myasthenia Gravis by toxin-acetylcholine receptor conjugates.” Jour. Immun. 133:1335-2549; Jansen, F. K., H. E. Blythman, D. Carriere, P. Casella, O. Gros, P. Gros, J. C. Laurent, F. Paolucci, B. Pau, P. Poncelet, G. Richer, H. Vidal, and G. A. Voisin. 1982. “Immunotoxins: Hybrid molecules combining high specificity and potent cytotoxicity”. Immunological Reviews 62:185-216; and Vitetta et al., supra).

Preferred linkers are described in the literature. See, for example, Ramakrishnan, S. et al., Cancer Res. 44:201-208 (1984) describing use of MBS (M-maleimidobenzoyl-N-hydroxysuccinimide ester). See also, Umemoto et al. U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pyridyl-dithio)toluene (Pierce Chem. Co., Cat. (21558G); (ii.) SPDP (succinimidyl-6 [3(2-pyridyldithio) propionamido] hexanoate (Pierce Chem. Co., Cat #21651G); (iv) Sulfo-LC-SPDP (sulfosuccinimidyl 6 [3-(2-pyridyldithio)propianamide] hexanoate (Pierce Chem. Co. Cat. #2165-G); and (v) sulfo-NHS(N-hydroxysulfo-succinimide: Pierce Chem. Co., Cat. #24510) conjugated to EDC.

The linkers described above contain components that have different attributes, thus leading to conjugates with differing physiochemical properties. For example, sulfo-NHS esters of alkyl carboxylates are more stable than sulfo-NHS esters of aromatic carboxylates. NHS ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

These antibodies may also be used as carriers to form immunotoxins. As such, they may be used to deliver a desired chemical or cytotoxic moiety to cell expressing the receptor. The cytotoxic moiety of the immunotoxin may be a cytotoxic drug or an enzymatically active toxin of bacterial, fungal or plant origin, or an enzymatically active polypeptide chain or fragment (“A chain”) of such a toxin. Enzymatically active toxins and fragments thereof are preferred and are exemplified by diphtheria toxin A fragment, non-binding active fragments of diphtheria toxin, exotoxin A (from Pseudomonas aer-uginosa), ricin A chain, abrin A chain, modeccin A chain, alphasarcin, certain Aleurites fordii proteins, certain Dianthin proteins, Phytolacca americana proteins (PAP, PAPII and PAP-S), Momordica charantia inhibitor, curcin, crotin, Saponaria officinalis inhibitor, gelonin, mitogellin, restrictocin, phenomycin, and enomycin, Ricin A chain, Pseudomonas aeruginosa exotoxin A and PAP are preferred.

Conjugates of the monoclonal antibody and such cytotoxic moieties may be made using a variety of bifunctional protein coupling agents. Examples of such reagents are N-succinimidyl-3-(2pyridyldithio) propionate (SPDP), iminothiolane (IT), bifunctional derivatives of imidoesters such as dimethyl adelpimidate HCl, active esters such as disuccininidyl suberate, aldehydes such as glutaradehyde, bis-azido compounds such as bis(p-diazoniumbenzoyl)ethylenediamine, diisocyanates such as tolylene 2,6-diisocyante, and bisactive fluorine compounds such as 1,5-difluoro-2,4-dinitrobenzene.

The enzymatically active polypeptide of the immunotoxins according to the invention may be recombinantly produced. Recombinantly produced ricin toxin A chain (RRTA) may be produced in accordance with the methods disclosed in PCT WO85/03508 published Aug. 15, 1985. Recombinantly produced diphtheria toxin A chain and non-binding active fragments thereof are also described in PCT WO85/03508 published Aug. 15, 1985.

Antibodies of the present invention can be detected by appropriate assays, e.g., conventional types of immunoassays. For example, a sandwich assay can be performed in which the receptor or fragment thereof is affixed to a solid phase. Incubation is maintained for a sufficient period of time to allow the antibody in the sample to bind to the immobilized polypeptide on the solid phase. After this first incubation, the solid phase is separated from the sample. The solid phase is washed to remove unbound materials and interfering substances such as nonspecific proteins which may also be present in the sample. The solid phase containing the antibody of interest bound to the immobilized polypeptide of the present invention is subsequently incubated with labeled antibody or antibody bound to a coupling agent such as biotin or avidin. Labels for antibodies are well-known in the art and include radionuclides, enzymes (e.g. maleate dehydrogenase, horseradish peroxidase, glucose oxidase, catalase), fluors (fluorescein isothiocyanate, rhodamine, phycocyanin, fluorescamine), biotin, and the like. The labeled antibodies are incubated with the solid and the label bound to the solid phase is measured, the amount of the label detected serving as a measure of the amount of anti-urea transporter antibody present in the sample. These and other immunoassays can be easily performed by those of ordinary skill in the art.

The following Examples serve to illustrate the present invention, and are not intended to limit the invention in any manner.

### EXAMPLES

### Example #1

### The cDNA Library Construction and Cloning of the CEA-Binding Protein

To isolate the CEA receptor a rat KC cDNA library was constructed using the MAP Express vector system (Stratagene, La Jolla, Calif.). The library was screened with the mouse polyclonal anti-80 kD antibody (11). By screening of 10¹¹ plaques one positive cDNA clone with an insert size of 2.4 kb was isolated. Sequencing and a Blast search of human genome data bases for close related family members revealed that this cDNA represents a novel rodent gene with 91% of homology to human hnRNP M4 protein.

The hnRNPs are a large group of over 20 proteins (hnRNP A-hnRNP U) that associate with pre-mRNAs in eukaryotic cell (12). The majority of hnRNPs are extremely abundant nuclear components, their amounts approximately the same as those of the core histones (13). Most hnRNPs are expressed only in the nucleus. However, several, among them, hnRNP A, D, E, I, and K have been found to shuttle between the nucleus and the cytoplasm, and are believed to promote mRNA export by acting as adapters between mRNA and the transport machinery (14). In general, these proteins interact with RNA and are involved in mRNA processing and maturation (capping, splicing and polyadenilation. They can affect mRNA stability (15). Some of hnRNPs also bind single and double stranded DNA and act as transcription factors (16). The hnRNP M4 protein belongs to the family of hnRNP M proteins that consists of 4 splicing forms with the molecular weight 68-80 k) (17). The human hnRNP M4 protein has been shown to participate in RNA splicing and processing, as well as to changes related with heat shock (18, 19, 20). Evidence that this protein can be expressed on the cell surface comes from the fact that the close homologue of human hnRNP M4 was also described as a monomer of N-acetylglucosamine-specific receptor of the thyroid hormone NAGR1 (21). Later studies showed that this receptor identical to the hnRNP M4 (22).

The differences between the isolated rat and full-length human hnRNP M4 cDNA (# NM 005968. 1) are mostly single substitutions of nucleotides throughout the sequence. However, the significant difference between the isolated rat and full-length human hnRNP M4 cDNA (# NM 005968. 1) is a deletion of 117 nucleotides in a spacer region between two N-terminal RNA-binding domains (RBD-I and RBD-2). Similar deletion is also characteristic to Homo sapiens hnRNP M4 protein deletion mutant cDNA (# AF 061832). The new clone has been referred to as a rat homologue of human hnRNP M4 protein. The cDNA sequence was submitted to the Gene Bank (# U32577).

Since the anti-80 KD antiserum used to probe the rat KC library is polyclonal, there is potential to recognize the nonspecific protein epitopes. Independently, we screened for the CEA binding proteins using the yeast two-hybrid system (HybriZAP-2.1 Two-Hybrid Predigested Vector System, Stratagene, La Jolla, Calif.). As a bait, an engineered fragment of the full length CEA, containing a 146 amino acid (aa) fragment surrounding the PELPK binding site was used. In addition to rat Kupffer cell cDNA library, a commercial human liver cDNA library (Stratagene, La Jolla, Calif.) was acquired as a target library for the yeast two-hybrid system. As a result of interaction, a single cDNA clone was isolated that was identical in sequence to the human hnRNP M4 protein. Thus, both screening techniques, antibody probing and the yeast two-hybrid system, gave the same result indicative of a new functional role for the hnRNP M4 protein as the CEA-binding protein in KC.

### Example #2

### Analysis of Rat hnRNP M4 Homologue cDNA

The cloned rat cDNA is a 2351 nucleotides long full-length sequence with the polyadenylation signal AATAAA and a termination polyA tail. SEQ ID NO: 1 (shown in FIG. 1) is the cDNA sequence of rat homologue of human hnRNP M4.

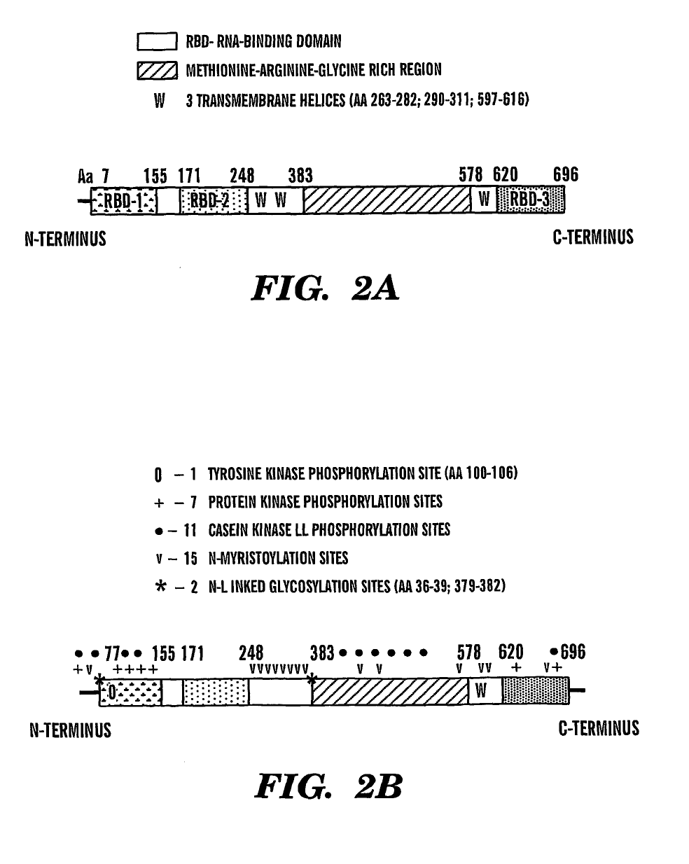

The sequence consists of a 5′ end nontranslated region of 21 bp and an open reading frame (ORF) that starts from the initiation sequence AAAATGG. The cDNA contains 3 putative RNA-binding domains (RBD). It is an evolutionarily conserved domain present in pre-mRNA-, mRNA-, pre-rRNA-, and snRNA-binding proteins, including hnRNP proteins, splicing factors, and polyadenylation factors (12). The first and second domains (RBD-1 and RBD-2) are arranged in a tandem close to the N-terminus and RBD-3 locates near the C-terminus (FIGS. 1 and 2a). It was shown that RBD motifs are characteristic of the RNA binding proteins and can provide RNA and DNA binding activity (16). N-terminal RBD-1 region (nucleotides 297-376) has. 87% of homology with Homo sapiens myelin gene expression factor 2 (MyEF-2) cDNA (25). The RBD-2 region (nucleotides 634-740) has 82% of identity to MyEF-2.

The MyEF-2 is a transcription factor that was isolated from mouse brain, maps to mouse chromosome 2 and represses transcription of myelin basic protein gene. The MyEF-2 protein contains two RNA-binding domains (RBD-1 and RBD-2), previously shown to be responsible for sequence specific binding to both RNA and single stranded DNA (25). RBD-1 and RBD-2 demonstrate a lesser degree of homology to RBDs found in a wide variety of RNA and single strand DNA binding proteins (26). Sequence analysis demonstrated that, hnRNP M4 contains no other commonly recognized DNA binding motifs, such as zinc finger, homeobox, POU, or helix-loop-helix domain. Presumably, similar to MyEF-2, RBDs can be responsible for the single strand DNA binding activity of hnRNP M4 and their presence suggests that this protein may also bind RNA.

The rat M4 protein sequence has 90-92% identity to a Homo sapiens chromosome 19 clone CTD-3182G2, complete sequence. Human gene by in situ hybridization was previously assigned to subbands p13.3-p13.2 of chromosome 19 (18, 21). The localization of the hnRNP M4 in the rat genome is not known.

### Example #3

### Analysis of Rat hnRNP M4 Protein

Sequence analysis of rat hnRNP M4 indicates that it encodes a novel protein of 775 amino acids (aa) consistent with its apparent molecular mass in SDS-polyacrylamide gel electrophoresis of 78-kD-80 kD. The protein is composed of 3 putative RNA-binding domains (aa 77-155, 171-248, 620-696). It has 3 potential spanning regions (aa 263-282, 290-311 and 597-616) (FIG. 2a). The intracellular domain contained what appeared to be a tyrosine phosphorylation motif (aa 100-106) and two glucosaminoglycan attachment sites (aa 36-39), (aa 379-382). These chains can serve to orient the receptor proteins on the cell surface by correct insertion into the plasma membrane (27) and to stabilize the protein (28).

Based on the translation of the primary sequence rat hnRNP M4 is a multifunctional signaling protein that can be modified by variety of enzymes. Structural analysis of the protein using Swiss-Prot database revealed that the protein possesses 7 potential protein kinase C phosphorylation sites, 11 casein kinase II phosphorylation sites and 15 N-myristoylation sites throughout the sequence (FIG. 2b).

The C-terminus contains 17 repeats of glycine-, arginine-, methionine and glutamine. Arginine methylation modification is a part of the mechanism by which protein-RNA complexes are recognized for nuclear export (29). Arginine is the only known methylated amino acid residue in hnRNPs. In fact, hnRNPs contain about 65% of methylated arginines in the cell nucleus (30), indicating that the methylation of these proteins is likely to have an important effect on their functions. It can influence the interactions of hnRNP M4 with other proteins (19) and affect its RNA-binding activity (31).

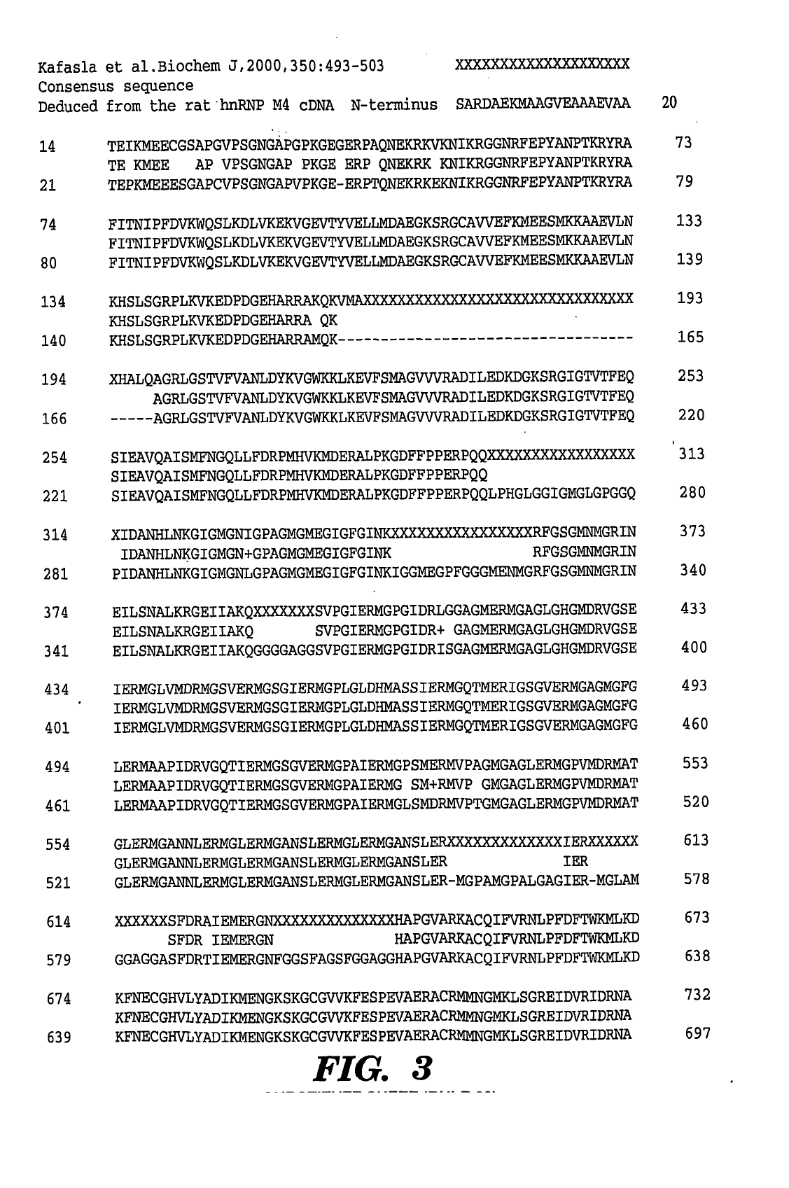

This data demonstrates that the hnRNP M4 protein contains several important structural motifs that are characteristic to receptors and molecules involved in the signaling transduction pathways. In support of this hypothesis is the partial protein sequence of the rat hnRNP M4 that has been recently published (17). We compared the two protein sequences and found 74% homology between them (FIG. 3, SEQ ID NO: 2). In contrast to our data, the published rat hnRNP M4 protein represents a partial sequence that is missing the N-terminal part of the protein. It also represents a splicing form without the 158-197 deletion in a spacer region between RBD-1 and RBD-2.

### Example #4

### In Vitro CEA Binding Studies

The ability of hnRNP M4 to interact with CEA was examined by preparing membrane lifts from 7ZAP Express-hnRNP M4 phage plates and incubating them with ¹²⁵I labeled CEA or PELPK albumin conjugate in the presence of 10 mM Ca⁺⁺ or 10 mM EDTA. Both CEA and PELPK albumin bound strongly to the phages in the presence of Ca⁺⁺ but there was no binding when EDTA was present. The data confirms our observations on the Ca⁺⁺ requirements of the CEA binding protein using isolated Kupffer cells and adds further evidence that hnRNP M4 is the Kupffer cell CEA binding protein.

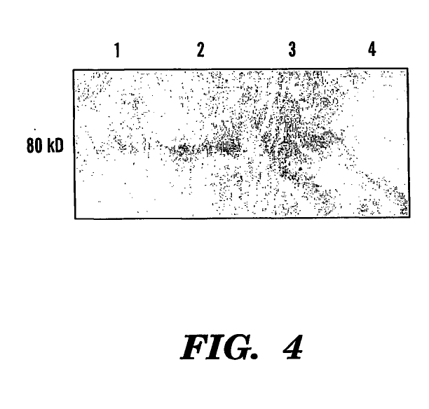

To further substantiate this finding and to estimate the molecular mass of the encoded protein, the cDNA corresponding to hnRNP M4 ORF was placed into a prokaryotic expression pGEX system allowing production of a GST-hnRNP M4 fusion protein. A series of pGEX vectors were designed for inducible, high level intracellular expression of the cDNA as a fusion protein with Schistosoma japonicum GST protein (Amersham Pharmacia Biotech, Piscataway, N.J.). The hnRNP M4 cDNA was inserted into pGEX-4T-3 vector to maintain the proper reading frame. The fusion protein was obtained as described below. The Kupffer cell lysate and pGEX-4T-3/hnRNP M4 fusion proteins (2 clones) were subjected to SDS/PAGE and transferred to a PVDF membrane (FIG. 4). This membrane was exposed to the soluble 1 μg/ml CEA followed by an anti-CEA antibody. KC lysate was used as a positive control (line 1) and the empty vector as a negative (line 4) control. A single 80 kD band was determined in Kupffer cells (line 1) and in the fusion proteins (lines 2, 3). This band was absent for the vector alone (line 4). This data indicated that rat hnRNP M4 protein encoded by the cDNA is able to bind soluble CEA similar to the 80 kD KC receptor. The molecular weights of the hnRNP M4 protein and an 80 KD receptor from KC were indistinguishable.

### Example #5

### Tissue Distribution of hnRNP M4

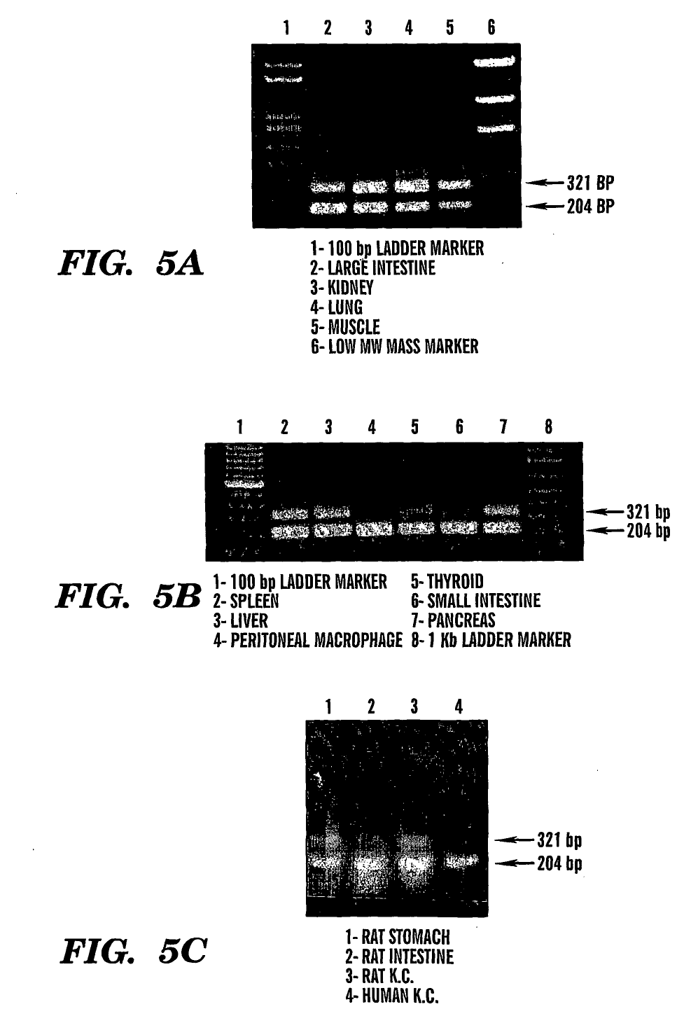

Both rat and human tissues were examined for the presence of the binding protein mRNA using Northern blots. A nick translated probe with [³²P] corresponding hnRNP M4 cDNA was used. The results from rat and human tissues were similar. Northern blot analysis revealed that the mRNA encoding hnRNP M4 is abundantly expressed as a single transcript of 2.4 kb in the liver, heart, lung, skeletal muscle, kidney, and stomach. The RNA seems to have a ubiquitous distribution but the Northern blot analysis was unable to distinguish between the full length and deletion mutant. To determine whether the form with the deletion in a spacer region between the RBD-1 and RBD-2 is expressed in Kupffer cells we synthesized PCR primers that incorporated the deleted region as part of their product. Amplification results in a larger PCR product for the full-length form (321 bp) and a shorter product (204 bp) for the deletion mutant, as illustrated in FIG. 5.

The results show that the deletion form encoding amino acids 158-197 of hnRNP M4 is distributed throughout all rat tissues examined. The larger form is generally also present but in what seems to be lower copy numbers. Comparison of the cDNA sequences indicates that there are multiple subclasses of mRNA that arise by alternative pre-mRNA splicing. Without wishing to be bound by theory, these results indicate that multiple forms of the hnRNP M4 protein may exist, possibly with different functions in vivo. Both mRNA forms (with and without the deletion) were characteristic to the rat KC. Only the short form was found in human KC. PCR results suggest that the short form of hnRNP M4 is exclusively expressed in human and is present in rat KC and can function as a CEA receptor. This data further supports our finding that the deleted forms of rat and human hnRNP M4 were isolated as CEA binding proteins.

### Example #6

### Transfection of hnRNP M4 in Macrophage Cell Lines

To elucidate whether the macrophage cell lines can uptake soluble CEA, in vitro experiments with ¹²⁵I labeled CEA were performed. We examined several macrophage cell lines: CRL2192, P388D, IC21, PH1 and raw 264.7. CEA uptake by isolated rat KC was used as a positive control. Cells were incubated with the iodine labeled CEA (5 μg/ml) for 15, 30, 45 and 60 minutes. None of the macrophage cell lines was able to take up labeled CEA in vitro. We have shown earlier that freshly isolated lung alveolar macrophages express the 80 kD protein and rapidly endocytose CEA in a similar manner to KC (32). These data suggest that the expression of the 80 kD CEA receptor and the ability to uptake CEA are cell specific and perhaps are tissue restricted to KC and alveolar macrophages.

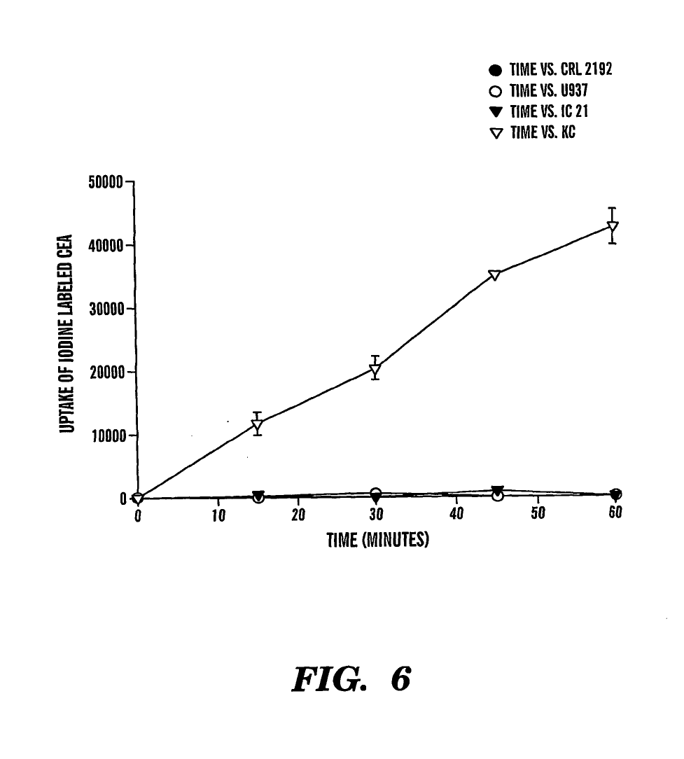

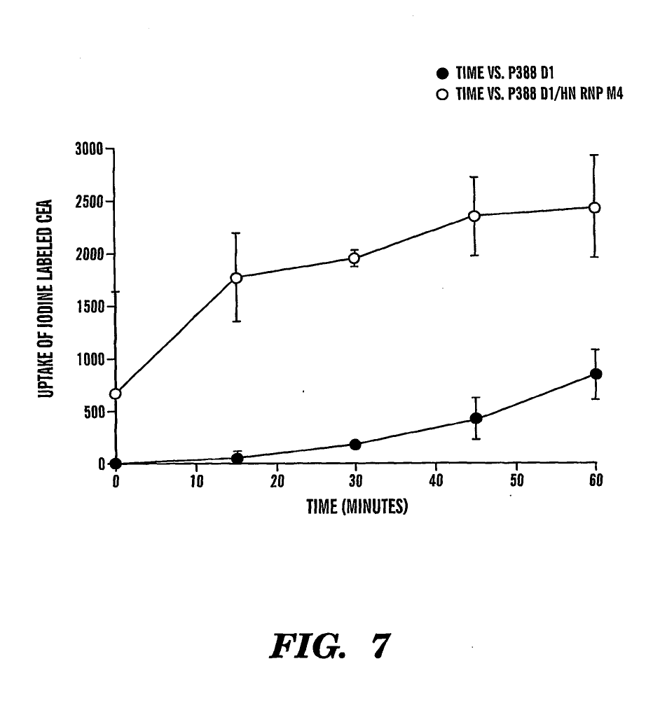

To begin to elucidate the mechanism underlying the intracellular signaling associated with KC activation and CEA binding, the hnRNP M4 cDNA in the pBK-CMV expression vector was introduced into CEA non-responsive macrophage cell lines. The hnRNP M4 transcription in this system was driven by the CMV promoter. The mouse alveolar macrophage: P388D1 and mouse peritoneal macrophage: IC21 cell lines that do not bind CEA and do not express CEA receptor were transfected with the hnRNP M4/PBK-CMV expression vector. As shown in FIG. 6, in transient transfection after 48 hours, the hnRNP M4 induced CEA binding in P388D1 cells was increased with increasing time of exposure and concentration of the hnRNP M4 plasmid DNA as compared with the co transfection of matching concentration of the vector. An approximately 5 times increase in CEA uptake was observed on transfecting 10 g of hnRNP M4/pBK-CMV plasmid. This fact implies that hnRNP M4 cDNA can initiate CEA binding in P388D1 alveolar macrophage cell line and can act as a receptor for CEA. Interestingly, only P388D1, but not IC21 cells were able to take up CEA (FIG. 7). At present, the reasons for lack of response by IC21 cells are not known.

### Example #7

### RNA Preparation and Analysis

The total RNA from various tissues, KC and cell lines was isolated using RNAzol method (Biotex Laboratories, Inc., Houston, Tex.) and the mRNA using mRNA isolation kit (Qiagen Inc., GmbH, Germany). Approximately 20 μg of total RNA were size fractionated on a formaldehyde/agarose gel, stained with ethidium bromide, and then transferred into nylon membrane (Hybond N, Amersham). HnRNP M4 probes were labeled by nick translation with [³²P]-dCTP and hybridized at a concentration of 2.10.6 cpm/ml at 65° C. in a solution containing 5×SSC, 2× Denhardt's solution, 0.1% SDS and 0.3 mg/ml salmon sperm DNA. The filters were washed under high stringency conditions (65° C. 0.1×SSC-0.1% SDS) prior to autoradiography.

### Example #8

### Rat cDNA Library Construction and Screening

Rat KC library was constructed on the basis of the ZAP Express vector system (Stratagene, La Jolla, Calif.) according to the manufacturer's instructions. In brief, bacterial strain XL1 was incubated with recombinant phages. Approximately 1.10⁶ pfu, of the library were plated on 150 mm Luria-Bertini agar plates containing 10 mM MgSO₄ and maintained at 42° C. for 2-3 hours. The PVDF filters pretreated in 10 mM IPTG were overlaid in the plates and incubated at 37° C. for 12 hours. Then the filters were dried and blocked in 3% milk prior to exposure to the anti-80 kD antibody (1:100 dilution). Enhanced chemiluminescence technique was used to detect positive cDNA clones (Amersham, Pharmacia). By screening 1011 phages one positive clone was identified, and after plaque purification, the insert was subcloned in pBK-CMV vector for DNA sequencing.

### Example #9

### DNA Sequencing

The sequencing was carried out using dideoxy terminator fluorescent DNA sequencing method with the Big Dye Terminator Cycle Sequencing Kit (Applied Biosystems). Reactions were purified by ethanol precipitation and separated on the ABI 377-96 slab gel automated DNA sequencer. The contingency sequence was assembled and analysed using the Vector NTI Suite program (Informax, Gathersburg, Mass.). The sequences were verified by sequencing of the opposite strand.

### Example #10

### Isolation of Kupffer Cells

Kupffer cells were isolated according to our standard laboratory protocol (11) from the livers of male Sprague Dawley rats or Balb/C mice. The rodents are starved overnight and the livers perfused through the portal vein with collagenase. Suspended cells are separated into parenchymal and non-parenchymal fractions by differential centrifugation. Kupffer cells are purified from the non-parenchymal cell fraction by using 17.5% solution of metrizamide in Gey's balanced salt solution for final separation. The interface layer containing Kupffer cells is isolated and washed in phosphate buffer. Further purification of the cells is achieved by attachment to plastic plates for 2 hr. Generally 3-5×10⁸ cells are routinely isolated from one liver. Kupffer cells are identified by their ability to phagocytose latex particles, staining for endogenous peroxidase activity and by electron microscopy.

### Example #11

### Western Blot Analysis

Cells (1×10⁷) were washed in PBS and then lysed in RIPA buffer (Santa Cruz, Calif.) with protease inhibitors (1 mM Sodium Orthovanadate, 2 mM Sodium Fluoride, 10 μg/ml leupeptin, and 0.5 mM PMSF). Cell lysates were centrifuged at 15,000 rpm for 10 minutes at 4° C. Supernatants were collected and protein concentration was measured by BCA kit (Pierce, Rockford, Ill.). Equal amount of protein was loaded on SDS-PAGE. After electrophoresis with Mini-Protein II apparatus at 200V for 50 min, samples were transferred into Sequi-Blot-PVDF nitrocellulose membrane (Bio-Rad Laboratories, Hercules, Calif.) for 1 hr at 100V. The membrane was incubated with the primary antibody (in concentration 1:250-1:500) dissolved in 3% milk/TBS blocking buffer overnight at 4° C. or for 1 hr at room temperature. Subsequently it was washed 3 times for 10 min with 1×TBS buffer and incubated with the corresponding secondary HRP antibody at 1:1-10,000 dilution for 1 hour at room temperature. After washing with 1×TBS buffer for 3 times (15 min each) at room temperature, detection of proteins of interest was carried out by ECL (Amersham, Life Science Inc, Cleveland, Ohio).

### Example #12

### Prokaryotic Protein Expression

For production of CEA binding protein in a bacterial system, the EcOR1/Xho1 fragment corresponding to hnRNP M4 cDNA from the recombinant phage was cloned into the EcOR1/Xho1 sites of pGEX-4T-3 vector (Amersham Pharmacia Biotech, Piscataway, N.J.). The pGEX-4T-3 backbone was chosen from the variety of pGEX-GST vectors to maintain the original reading frame. The plasmid was introduced into B21 E. coli host bacteria and the transformed cells were cultured in L-broth to OD600=0.5 after which IPTG (0.3 mM) were added and cultures were maintained at 37° C. for 2 hours. Total bacterial proteins were prepared and fusion proteins were purified using glutathione Sepharose 4B affinity chromatography, according the manufacturer's instructions (Amersham Pharmacia Biotech, Piscataway, N.J.).

### Example #13

### RT-PCR

Amplifications were performed as per recommended protocols by the suppliers of the reagents. Briefly, RNA was isolated from cell lines, embryos and tissues using a single step RNA isolation method as described above. Typically, 1 g of total RNA was used for reverse transcription reaction using M-MLV reverse transcriptase and random hexamer or oligo dT primers (Promega Corp., USA) in a 50 l reaction. One tenth of the RT-products were used for PCR amplifications using Taq polymerase (Promega Corp., USA; Life Technologies, USA) along with gene specific primer pairs employing various cycling parameters depending on the primer combinations (described in the relevant section). The PCR reaction products were analyzed on agarose gels by ethidium bromide staining and photographed using a gel documentation set up (Kodak digital science, USA). For isolation of the PCR products, the appropriate bands containing DNA were excised from the agarose gels and purified using gel extraction kits (Qiagen, GmbH, Germany). The purified DNA fragments were dissolved in TE (pH 7.6) and used for direct sequencing and/or subcloning into plasmid vectors.

### Example #14

### In Vitro CEA Binding Assay and CEA ¹²⁵I Labeling

These procedures were performed as described previously (10).

### Example #15

### Transient Transfection of Expression Plasmids into Macrophage Cell Lines

Transient transfection of hnRNP M4 plasmid was carried out in P388D1 mouse lymphoid macrophage, IC21 mouse peritoneal and CRL2192 rat alveolar macrophage cell lines. As a positive control were used rat KC. The P388D1 cells were propagated in DMEM medium supplemented with 10% horse serum. The CRL 2192 cells were grown in F-12K medium (Kaighn's modification) supplemented with 15% FBS. The IC21 cells were grown in RPM1 1640 ATCC modified medium supplemented with 10% FBS. All cells were maintained at 37° C. in 5% CO₂ atmosphere. All tissue culture products were obtained from Life Technologies, USA. Cells for transfection were plated at 2-2.5.10⁵ cells per well in six well tissue culture clusters (Nunc products, Germany). Cells were allowed to grow for 24 h and plasmid construct was transfected in serum free medium using Gene Pulser II Electroporation system (250 μF-300V) (Bio-Rad, Richmond, Calif., USA). The cells were washed once with PBS and resuspended at a density of 1.10.7 cells/ml in RPM1 media without FBS. 10 μg of pBK-CMV/hnRNP M4 expression vector DNA was transfected per sample. 0.4 ml of the cell suspension was used per electroporation in 0.4 cm cuvettes. The cells were maintained at room temperature prior to and after electroporation. At 10 minutes post electroporation, the cells are placed into growth media. At 48 hours post electroporation, the cells are harvested and resuspended in a volume of 200 μl for measuring of CEA binding activity.

The references cited herein and set forth below are incorporated herein in their entirety.

### REFERENCES

1. Kim J. C., Gong G., Roh S. A., Park K. C. Carcinoembryonic antigen gene and carcinoembryonic antigen expression in the liver metastasis of colorectal carcinoma. Mol Cells. 1999 Apr. 30; 9(2):133-7.
2. Majuri M. L., Hakkarainen M, Paavonen T, Renkonen R. Carcinoembryonic antigen is expressed on endothelial cells. A putative mediator of tumor cell extravasation and metastasis. APMIS. 1994; 102: 432-438.
3. Keesee S. K., Briggman J. V., Thill G., and Ying-Jye Wu, Utilization of nuclear matrix proteins for cancer diagnosis. Crit. Rev. Eukar. Gene. Expres., 1996; 6: 189-214.
4. Wagner H. E., Toth C. A., Steele G. D. and Thomas P. Invasive and metastatic potential of human colorectal cell lines: relationship to cellular differentiation and carcinoembryonic antigen production. Clin. Exp. Metastasis; 1992 10: 25-31.
5. Hashino J., Pukada Y., Oikawa S., Nakazato H., and Nakanishi T., Metastatic potential of human colorectal carcinoma SW1222 cells transfected with cDNA encoding carcinoembryonic antigen. Clin. Exp. Metastasis; 1994, 12: 324-328.
6. Thomas P, Gangopadhyay A, Steele G Jr, Andrews C, Nakazato H, Oikawa S, Jessup J M. The effect of transfection of the CEA gene on the metastatic behavior of the human colorectal cancer cell line MIP-101. Cancer Lett. 1995; 25: 59-66.
7. Leconte A, Garambois V, Ychou M, Robert B, Pourquier D, Terskikh A, Mach J P, Pelegrin A. Involvement of circulating CEA in liver metastases from colorectal cancers re-examined in a new experimental model. Br J Cancer. 1999, 9:1373.
8. Toth C. A. Liver endocytosis and Kupffer cells. Hepathology; 1992, 16: 255-266.
9. Toth C. A, Thomas P., Broitman S. A. and Zamcheck. A new Kupffer cell receptor mediating plasma clearance of carcinoembryonic antigen by the rat. Biochem. J; 1982, 204: 377-381.
10. Gangopadhyay A, Thomas P. Processing of carcinoembryonic antigen by Kupffer cells: Recognition of a penta-peptide sequence. Arch Biochem Biophys. 1996, 334: 151-157.
11. Gangopadhyay A, Lazure D A, Kelly T M, Thomas P. Purification and analysis of an 80-kDa carcinoembryonic antigen-binding protein from Kupffer cells. Arch Biochem Biophys. 1996, 328: 151-157.
12. Gorlach M., Burd C. G., Portman D. S., and Greyfuss G. HnRNP proteins. The Mol. Biol. Reports; 1993, 18: 73-78.
13. Huang S. Review: perinucleolar structures. J Struct. Biol. 2000 April 29(2-3):233-40.
14. M, Jang S K, Shyu A B, Wilkinson M F. The double lives of shuttling mRNA binding proteins. Cell. 2000; 102:135-138.
15. Loflin P, Chen C Y, Shyu A B. Unraveling a cytoplasmic role for hnRNP D in the in vivo mRNA destabilization directed by the AU-rich element. Genes Dev. 1999; 13:1884-1897.
16. Tolnay M, Vereshchagina L A, Tsokos G C. Heterogeneous nuclear ribonucleoprotein DOB is a sequence-specific DNA-binding protein. Biochem J. 1999; 338 :417-425. )
17. Kafasla P, Patrinou-Georgoula M, Guialis A. The 72/74-kDa polypeptides of the 70-110 S large heterogeneous nuclear ribonucleoprotein complex (LH-nRNP) represent a discrete subset of the hnRNP M protein family. Biochem J.; 2000, 350: 495-503.
18. Gattoni R, Mahe D, Mahl P, Fischer N, Mattei M G, Stevenin J, Fuchs J P. The human hnRNP-M proteins: structure and relation with early heat shock-induced splicing arrest and chromosome mapping. Nucleic Acids Res. 1996; 24:2535-2542.
19. Datar K. V., Dreyfuss G., Swanson M. S. The human hnRNP M proteins: identification of a methionine/arginine rich repeat motif in ribonucleoproteins. Nucl. Acids Res., 1993, 21: 439-446.
20. Mahe D, Fischer N, Decimo D, Fuchs J. Spatiotemporal regulation of hnRNP M and 2H9 gene expression during mouse embryonic development. Biochim Biophys Acta. 2000, 1492: 414-424.
21. Blank O., Perrin C., et. al., Molecular cloning, cDNA analysis, and localization of a monomer of the N-acetylglucosamine-specific receptor of the thyroid, NAGR1, to chromosome 19p13.3.-13.2. Genomics 1994, 21: 18-26.
22. Blanck O, Perrin C, Mziaut H, Darbon H, Mattei MG, Miquelis R. Molecular cloning, cDNA analysis, and localization of a monomer of the N-acetylglucosamine-specific receptor of the thyroid, NAGR1, to chromosome 19p13.3-13.2. Genomics. 1995, 27: 561.
23. Gangopadhyay A., Lazure D. A., Thomas P. Carcinoembryonic antigen induces signal transduction in Kupffer cells. Cancer Lett. 1997; 118: 1-6.
24. Gangopadhyay A., Bajenova O., Kelly T. M., and Thomas P. Carcinoembryonic antigen induces cytokine expression in Kupffer cells: Implications for hepatic metastasis from colorectal cancer. Cancer Research, 1996; 56: 4805-4810.
25. Haas S., Steplewski A., Siracusa L. D., Amini S., Khalili K. Identification of a sequence-specific single-stranded DNA binding protein that suppresses transcription of the mouse myelin basic protein gene. J. Biol. Chem. 1995, 270: 12503-12510.
26. Kenan D. J., Query C. C. and Keene J. D. RNA recognition: towards identifying determinants of specificity. (1991) Trends Biochem. Sci 16:214-220.
27. Boer U., Neuschafer-Rube F, Moller U., Puschel G. P. Requirement of N-glycosylation of the prostaglandin E2 receptor EP3beta for correct sorting to the plasma membrane but not for correct folding. Biochem J., 2000; 15: 839-847.
28. Rudd P. M., Wormald M. R., Dwek R. A., Roles of glycosylation of cell surface receptors involved in cellular immune recognition. J. Mol. Biol. 1999; 239: 351-366.
29. Liu Q., Dreyfuss G. In vivo and in vitro arginine methylation of RNA-binding proteins. Mol Cell Biol. 1995 May; 15(5):2800-8.
30. Shen E. C., Henry M. F., Weiss V. H., Valentini S. R., Silver P. A., Lee M. S. Arginine methylation facilitates the nuclear export of hnRNP proteins. Genes Dev. 1998; 12: 679-91.
31. Rajpurohit R., Paik W. K., Kim S. Effect of enzymic methylation of heterogeneous ribonucleoprotein particle A1 on its nucleic-acid binding and controlled proteolysis. Biochem J. 1994 Dec. 15; 304 (Pt 3):903-9.
32. Toth C. A., Rapoza A., Zamcheck N., Steele G., Thomas P. Receptor mediated endocytosis of carcinoembryonic antigen by rat alveolar macrophages in vitro. J. Leukocyte Biology. 1989; 45: 370-376.
33. Schullery D.S., Ostrowski J., Denisenko O. N., Stempka L., Shnyreva M., Suzuki H., Gschwendt M., Bomsztyk K. Regulated interaction of protein kinase Cdelta with the heterogeneous nuclear ribonucleoprotein K protein. J. Biol. Chem. 1999; 274:15101-15109.
34. Bomsztyk K., Van Seuningen I., Suzuki H., Denisenko O., Ostrowski J. Diverse molecular interactions of the hnRNP K protein. FEBS Lett. 1997; 403:113-125.
35. Bosser R., Faura M., Serratosa J., Renau-Piqueras J., Pruschy M, Bachs O. Phosphorylation of rat liver heterogeneous nuclear ribonucleoproteins A2 and C can be modulated by calmodulin. Mol. Cell. Biol. 1995; 15: 661-670.
36. Kim J. H., Hahm B., Kim Y. K., Choi M., Jang S. K. Protein-protein interaction among hnRNPs shuttling between nucleus and cytoplasm. J. Mol. Biol.; 2000, 298: 395-405.
37. Kim J C, Kim W S, Ryu J S, Oh S J, Lee D H, Koo K. H., Roh S. A., Kim H. C., Yu C. S., Kang G. H., Bodmer W. F. Applicability of carcinoembryonic antigen-specific monoclonal antibodies t6 radioimmunoguided surgery for human colorectal carcinoma. Cancer Res., 2000, 60: 4825-4829.
38. Szalai G, Williams L. E., Primus F. J. Tumor targeting with radiolabeled antibodies in a human carcinoembryonic antigen transgenic mouse model. Int. J. Cancer, 2000; 85: 751-756.
39. Xu X., Clarke P., Szalai G., Shively J. E., Williams L. E., Shyr Y., Shi E., Primus. Targeting and therapy of carcinoembryonic antigen-expressing tumors in transgenic mice with an antibody-interleukin-2 fusion protein. FJ Cancer Res., 2000; 60: 4475-4484.
40. Nolan K. F., Yun C. O., Akamatsu Y., Murphy J. C., Leung S. O., Beecham E J, Junghans R. P. Bypassing immunization: optimized design of “designer T cells” against carcinoembryonic antigen (CEA)-expressing tumors, and lack of suppression by soluble CEA. Clin. Cancer; 1999; 5: 3928-3941.

1. A method of preventing tumor metastasis comprising administering to a host having a primary tumor a compound that inhibits binding of CEA to a hnRNP M4 receptor.

2. The method of claim 1, wherein the compound is an antibody.

3. The method of claim 1, wherein the compound is an antisense DNA.

4. The method of claim 1, wherein the compound is a soluble receptor.

5. An isolated DNA segment encoding a protein comprising the amino acids of SEQ ID NO: 2.

6. A DNA segment encoding an hnRNP M4 receptor having the amino acid of SEQ ID NO: 2.

7. An isolated DNA segment encoding a human hnRNP M4 receptor.

8. A protein encoded by the DNA of claim 5, 6 or 7.

9. An antibody directed to the protein of claim 8.

10. An isolated DNA segment comprising the nucleotide sequence of SEQ ID NO: 1, or the complement thereof.

11. A host cell containing the DNA of claim 5, 6 or 7.

12. A bioassay for detecting hnRNP M4 mRNA in a biological sample comprising the steps of:
i) contacting said biological sample with a DNA segment according to claims 5, 6 or 7 under conditions such that a DNA:RNA hybrid molecule containing said DNA segment and complementary RNA can be formed; and
ii) determining the amount of said DNA segment present in said hybrid molecule.

13. A bioassay for testing potential analogs of ligands of hnRNP M4 receptors for the ability to affect an activity mediated by said hnRNP M4 receptors, comprising the steps of:
i) contacting a molecule suspected of being a ligand with hnRNP M4 receptors produced by a cell according to claim 11; and
ii) determining the amount of a biological activity mediated by said hnRNP M4 receptors in said cells.

14. An assay for detecting an hnRNP M4 in a biological sample comprising the steps of:
i) contacting said sample with an antibody according to claim 9, under conditions such that specific complexes of said antibody and an antigen can be formed; and
ii) determining the amount of said antibody present as said complexes.

15. A method for targeting a therapeutic drug to cells having high levels of hnRNP M4 receptors, comprising the steps of:
i) conjugating an antibody according to claim 9, or an active fragment thereof, to said drug; and
ii) administering the resulting conjugate to an individual with cells having high levels of hnRNP M4 receptors in an effective amount and by an effective route such that said antibody is able to bind to said receptors on said cells.

16. Use of antibody of claim 9, or an active fragment thereof, conjugated to a therapeutic drug to target said therapeutic drug to cells having high levels of hnRNP M4 receptors.
